(19) United States
(12) Patent Application Publication
(10) Pub. No.: US 2024/0410868 A1
(43) Pub. Date: Dec. 12, 2024

(54) ADAPTIVE ALARM THRESHOLDS FOR RATE OF CHANGE IN DISSOLVED GAS CONCENTRATION IN TRANSFORMER FOR FAULT DETECTION

(71) Applicant: GE Infrastructure Technology LLC, Greenville, SC (US)

(72) Inventors: Balakrishna PAMULAPARTHY, Hyderabad (IN); Palak JAIN, Hyderabad (IN); Hiteshkumar MISTRY, Bengaluru (IN); Shivanand BHAVIKATTI, Bengaluru (IN)

(73) Assignee: GE Infrastructure Technology LLC, Greenville, SC (US)

(21) Appl. No.: 18/736,595

(22) Filed: Jun. 7, 2024

(30) Foreign Application Priority Data
Jun. 9, 2023 (IN) 202341039728

(51) Int. Cl. G01N 33/00; H02M 1/00

(52) U.S. Cl. CPC G01N 33/0063; H02M 1/0003

(57) ABSTRACT

A method for using adaptive alarm thresholds for rate of change in dissolved gas concentrations for power transformer fault detection may include receiving first dissolved gas data of a power transformer; determining a first rate of change (ROC) of a first gas concentration of the first dissolved gas data; generating, based on the first ROC, a first adaptive alarm threshold for ROC with which to detect a fault in the power transformer; receiving second dissolved gas data of the power transformer; determining a second ROC of a second gas concentration of the second dissolved gas data; comparing the second gas concentration to a static gas concentration threshold; comparing, based on the comparison of the second gas concentration to the static gas concentration threshold, the second ROC to the first adaptive alarm threshold for ROC; detecting the fault based the comparisons; and generating an alert indicative of the fault.

### CROSS-REFERENCE TO RELATED APPLICATION(S)

This application claims the priority benefit of Indian application No. 202341039728, filed Jun. 9, 2023, which is incorporated herein, in its entirety, by reference.

### TECHNICAL FIELD

This disclosure generally relates to adaptive alarm thresholds for rate of change in dissolved gas concentration in transformer for fault detection.

### BACKGROUND

Transformer oil dissolved gas analysis is a useful, predictive, and effective way for evaluating transformer health. The breakdown of electrical insulating material and related components inside a transformer may generate gases that may be indicative of transformer faults, so detecting the concentration of gases generated and their rate of increase may allow for transformer maintenance by accurate and robust fault diagnosis and prognosis.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates an example transformer system using dissolved gas analysis with adaptive alarm thresholds for rate of change in dissolved gas concentration in accordance with one embodiment of the present disclosure.

FIG. 2 illustrates an example process for using dissolved gas analysis with adaptive alarm thresholds for rate of change in dissolved gas concentration in accordance with one embodiment of the present disclosure.

FIG. 3A illustrates an example process for computing adaptive alarm thresholds for rate of change in dissolved gas concentration in accordance with one embodiment of the present disclosure.

FIG. 3B illustrates the example methodologies of FIG. 3A for computing adaptive alarm thresholds for rate of change in dissolved gas concentration based on a variable window in accordance with one embodiment of the present disclosure.

FIG. 4 illustrates an example graph showing rate of change and delta rate of change of dissolved gas concentration used in dissolved gas analysis with adaptive alarm thresholds for rate of change in dissolved gas concentration in accordance with one embodiment of the present disclosure.

FIG. 5 is a diagram illustrating an example machine that may be used in implementing embodiments of the present disclosure.

Certain implementations will now be described more fully below with reference to the accompanying drawings, in which various implementations and/or aspects are shown. However, various aspects may be implemented in many different forms and should not be construed as limited to the implementations set forth herein; rather, these implementations are provided so that this disclosure will be thorough and complete, and will fully convey the scope of the disclosure to those skilled in the art. Like numbers in the figures refer to like elements throughout. Hence, if a feature is used across several drawings, the number used to identify the feature in the drawing where the feature first appeared will be used in later drawings.

### SUMMARY

A method for using adaptive alarm thresholds for rate of change in dissolved gas concentrations for power transformer fault detection may include receiving, by at least one processor of a device, from at least one sensor of an power transformer, first dissolved gas data of the power transformer; determining, by the at least one processor, a first rate of change (ROC) based on a sliding time window of a first gas concentration of the first dissolved gas data where the length of time window can be fixed and variable determined by operator; generating, by the at least one processor, based on the first ROC, a first adaptive alarm threshold for ROC with which to detect a fault in the power transformer; receiving, by the at least one processor, from the at least one sensor, second dissolved gas data of the power transformer; determining, by the at least one processor, a second ROC based on a sliding time window of a second gas concentration of the second dissolved gas data where the length of time window can be fixed and variable determined by operator; comparing, by the at least one processor, the second gas concentration to a static gas concentration threshold; comparing, by the at least one processor, based on the comparison of the second gas concentration to the static gas concentration threshold, the second ROC to the first adaptive alarm threshold for ROC; detecting, by the at least one processor, the fault based the comparison of the second gas concentration and their ROC to the their alarm thresholds respectively-static or adaptive; and generating, by the at least one processor, an alert indicative of the fault; receiving, by the at least one processor, from the at least one sensor, subsequent dissolved gas data of the power transformer; determining, by the at least one processor, a subsequent ROC based on a sliding time window of a subsequent gas concentration of the subsequent dissolved gas data where the length of time window can be fixed and variable determined by operator; comparing, by the at least one processor, the subsequent gas concentration to a static gas concentration threshold; comparing, by the at least one processor, based on the comparison of the subsequent gas concentration to the static gas concentration threshold, the subsequent ROC to the previous subsequent adaptive alarm threshold for ROC; detecting, by the at least one processor, the fault based on the comparison of the subsequent gas concentration and their ROC to their alarm thresholds respectively-static or adaptive; and generating, by the at least one processor, an alert indicative of the fault.

A device for using adaptive alarm thresholds for rate of change in dissolved gas concentrations for power transformer fault detection may include memory coupled to at least one processor, the at least one processor able to: receive, from at least one sensor of an power transformer, first dissolved gas data of the power transformer; determine a first rate of change (ROC) based on a sliding time window of a first gas concentration of the first dissolved gas data where the length of time window can be fixed and variable determined by operator; generate, based on the first ROC, a first adaptive alarm threshold for ROC with which to detect a fault in the power transformer; receive, from the at least one sensor, second dissolved gas data of the power transformer; determine a second ROC based on a sliding time window of a second gas concentration of the second dissolved gas data where the length of time window can be fixed and variable determined by operator; compare the second gas concentration to a static gas concentration threshold; compare, based on the comparison of the second gas concentration to the static gas concentration threshold, the second ROC to the first adaptive alarm threshold for ROC; detect the fault based the comparison of the second gas concentration and their ROC to the their alarm thresholds respectively-static or adaptive; and generate an alert indicative of the fault; receiving, by the at least one processor, from the at least one sensor, subsequent dissolved gas data of the power transformer; determining, by the at least one processor, a subsequent ROC based on a sliding time window of a subsequent gas concentration of the subsequent dissolved gas data where the length of time window can be fixed and variable determined by operator; comparing, by the at least one processor, the subsequent gas concentration to a static gas concentration threshold; comparing, by the at least one processor, based on the comparison of the subsequent gas concentration to the static gas concentration threshold, the subsequent ROC to the previous subsequent adaptive alarm threshold for ROC; detecting, by the at least one processor, the fault based on the comparison of the subsequent gas concentration and their ROC to their alarm thresholds respectively-static or adaptive; and generating, by the at least one processor, an alert indicative of the fault.

A system for using adaptive alarm thresholds for rate of change in dissolved gas concentrations for power transformer fault detection may include: a dissolved gas analyzer device; and memory coupled to at least one processor able to: receive, from at least one sensor of an power transformer, first dissolved gas data of the power transformer; determine a first rate of change (ROC) based on a sliding time window of a first gas concentration of the first dissolved gas data where the length of time window can be fixed and variable determined by operator; generate, based on the first ROC, a first adaptive alarm threshold for ROC with which to detect a fault in the power transformer; receive, from the at least one sensor, second dissolved gas data of the power transformer; determine a second ROC based on a sliding time window of a second gas concentration of the second dissolved gas data where the length of time window can be fixed and variable determined by operator; compare the second gas concentration to a static gas concentration threshold; compare, based on the comparison of the second gas concentration to the static gas concentration threshold, the second ROC to the first adaptive alarm threshold for ROC; detect the fault based the comparison of the second gas concentration to the first adaptive alarm threshold for ROC; and generate an alert indicative of the fault; receiving, by the at least one processor, from the at least one sensor, subsequent dissolved gas data of the power transformer; determining, by the at least one processor, a subsequent ROC based on a sliding time window of a subsequent gas concentration of the subsequent dissolved gas data where the length of time window can be fixed and variable determined by operator; comparing, by the at least one processor, the subsequent gas concentration to a static gas concentration threshold; comparing, by the at least one processor, based on the comparison of the subsequent gas concentration to the static gas concentration threshold, the subsequent ROC to the previous subsequent adaptive alarm threshold for ROC; detecting, by the at least one processor, the fault based on the comparison of the subsequent gas concentration and their ROC to their alarm thresholds respectively-static or adaptive; and generating, by the at least one processor, an alert indicative of the fault.

### DETAILED DESCRIPTION

Dissolved Gas Analysis (DGA) is useful in detecting and predicting transformer faults. However, DGA may generate false positive alarms that incorrectly indicate a transformer fault based on the presence of gases in transformers. Alarm thresholds used to detect transformer fault based on gas levels often are set manually based on established standards. For example, the IEEE and IEC standards organizations have set fixed alarm thresholds for gas concentration and their rate of change (ROC), such as IEEE-C57.104-2019. For example, the IEEE-C57.104-2019 standard recommends static alarm thresholds that are generic based on its own transformer network or fleet historical data instead of those that are specific to a transformer. The IEC 60599-1999 standard provides the following alarm thresholds for transformer gas ROC, as shown in Table 1.

TABLE 1
IEC 60599-1999 Static Alarm Thresholds for
rate of change in gas concentration:

| Gas | Values in millimeters/day |
|---|---|
| Hydrogen | <5 |
| Methane | <2 |
| Ethane | <2 |
| Ethylene | <2 |
| Acetylene | <0.1 |
| Carbon monoxide | <50 |
| Carbon dioxide | <200 |

However, the static alarm thresholds may result in false positives and may not be robust for grids with penetration of distributed energy resources, decarbonization, different types of loads being added, different loading characteristics of transformers, and different transformer manufacturing. Fixed alarm thresholds used in the standards may be transformer-agnostic and based on the 90th/95th percentile of thousands of same types of transformers connected in a network. As a transformer ages or experiences significant or frequent load changes, the generated gas concentrations tend to rise naturally. A possible result is a false fault alarm based on the fixed alarm thresholds.

Some alarm threshold techniques use a gradient (ROC) calculation from the established standards (e.g., 95th percentile). The fixed threshold (or norms) from standards uses a statistical parameter of the very large amount of data which is obtained from the same types of devices in a network and is being collected over a very long period. That parameter is 90^{th} or 95^{th} percentile. However, setting a fixed threshold at deployment time may not be sufficient in some situations, as this threshold may need to evolve so that the system does not issue large numbers of false alarms.

In one or more embodiments, the transformer fault alarms may use adaptive thresholds for dissolved gas concentrations and their ROC for transformers in operation. The present disclosure provides multiple new techniques for setting and using adaptive alarm thresholds and for determining a gas concentration ROC. A dissolved gas analyzer may provide a gas concentration in parts-per-million (ppm), and from the gas concentration, the gradient (ROC) may be calculated. Adaptive alarm thresholds may be used for both the gas concentration and the ROC. The gas concentrations from DGA may be compared to static or adaptive alarm thresholds, and when the gas concentration exceeds a threshold, a probable fault condition may be hypothesized. To confirm the fault condition, their ROCs may be compared to the adaptive thresholds to minimize false alarms. In this manner, the fault detection and confirmation may be based on comparing both gas concentration and their gradient (i.e. ROC) to alarm thresholds (static or adaptive), with at least the ROC alarm thresholds being adaptive.

The benefits of the enhanced techniques of the present disclosure include reducing false positives of transformer fault detection alarms, improved anomaly detection, reducing the need to set alarm thresholds manually for transformers, and reducing computational expense (e.g., allowing for the techniques to be applied on a variety of devices). In contrast with some techniques that use an established (e.g., 95th percentile) ROC threshold, the enhanced adaptive thresholds herein may use prior knowledge from the fixed thresholds of the standards to determine the adaptive thresholds more adaptively. For reference, a fixed-size window t_{ω} may slide over a device data frame and its 95th percentile generates a threshold τ that may vary as the window slides: τ=f(β, t_{ω}) 95th percentile of βₜ_{_{ω}}, where τ is an adaptive threshold for ROC alarms. Even these adaptive thresholds are less than those from the standard and may result in higher false positives because the data used by the standards includes a variety of transformer health conditions, such as fault-free, incipient fault, sever fault, and breakdown, whereas the data used in the enhanced techniques herein has no fault condition.

In one or more embodiments, the enhanced techniques herein provide a data-driven methodology to detect accurate false conditions and minimize the false alarms via adaptive alarm threshold estimation. The proposed methodology enables a transformer-specific adaptive threshold unlike the transformer-agnostic ones from the standard that do not account for transformer age, power and voltage ratings, maintenance and overhauling, and other factors causing transformer degradation. The transformer-specific adaptive alarm threshold herein may be calculated using time-series gas concentration data generated by a DG Analyzer, and may be set by applying statistical analysis on recent transformer gas concentrations and their ROCs that have indicated no fault condition. The adaptive threshold may be compared with a new data point at sampling time, and a fault condition may be confirmed if the new data point is above the adaptive threshold. Otherwise, the transformer may be considered healthy, and the adaptive threshold may be updated with that data point. In this manner, the proposed methodology minimizes false alarms as it considers the recent transformer condition and complements fixed threshold methodology given in standards.

In one or more embodiments, a DG analyzer may sample gases in a transformer at any sampling rate (e.g., the enhanced techniques are agnostic to sampling rate). The DG analyzer may determine gas concentrations of respective gases and their ROCs. The ROC calculation may use Equation (1): p=βt+α, where p is the concentration (ppm) of a gas target measurement, β is the ROC formula, t is the time from initial zero start time, and α is a vertical axis intercept. After determining the ROC for a gas, the DG analyzer may apply one of the data-driven techniques disclosed herein to generate adaptive alarm thresholds for fault detection based on ROC. The DG analyzer may apply the adaptive alarm thresholds and the standard-based thresholds to the data stream of gas data to determine whether a subsequent ROC exceeds its adaptive alarm threshold.

In one or more embodiments, the DG analyzer may use ROC limits from the standard, that is prior knowledge of thresholds, and add them to an adaptive threshold. Using the example of Table 1 above, the standard-based Hydrogen alarm threshold is 5 mm/day. Alternatively, the DG analyzer may operate with no prior knowledge of thresholds from the standard to an adaptive threshold (e.g., ignore the 5 mm/day threshold for Hydrogen). With no prior knowledge of thresholds from the standard, the DG analyzer may generate an adaptive threshold for a gas ROC. The DG analyzer may use some previous ROC values for a gas using a fixed or variable sliding time window of ROC values (e.g., use the last n ROC values, or use a variable window of ROC values at each sampling rate). The variable time window reduces ROC computations unlike the case of fixed time window where a new ROC value is calculated at every sampling rate, The DG analyzer may increase or reduce the sliding time window size (i.e., the number of ROC values) when the DG analyzer detects no change or a change in gas concentration respectively. When the window of ROC samples is variable or fixed, the DG analyzer may use the L2 norm (or another norm) of the ROC or difference of two consecutive sorted ROCs referred to as Delta ROC, mu+α*SD, where mu is the mean and SD is standard deviation of the ROC or the Delta ROC and a is the multiplication factor with a default value of 2, or mu_w+SD_w with a weighted mu and weighted standard deviation. Some of the methodologies with a fixed window of ROC samples are shown below in Table 2.

TABLE 2
Fixed ROC Window (n is fixed) based Adaptive Alarm Thresholds:

| | Param- | Threshold With No Prior Knowledge |
|---|---|---|
| Method | eter | of Thresholds from the Standard |
| Mean and | ROC | Tₜ_{_{w}} = μ_{β} + 2 × ϑ_{β} |
| Standard | | |
| Deviation | | |
| Weighted Mean and Weighted Standard Deviation | ROC | Tₜ_{_{w}} = μ_{wβ} + 2 × ϑ_{wβ} ${μ}_{ωβ} = \frac{{\sum }_{i = 1}^{n} {ω}_{i} {β}_{{t}_{w}} ( i )}{{\sum }_{i = 1}^{n} {ω}_{i}}$ |
| | | ${ω}_{i} = 1 - \frac{\vert {μ}_{β} - {β}_{{t}_{w} ( i )} \vert}{max \vert {μ}_{β} - {β}_{{t}_{w}} \vert}$ |
| | | ${ϑ}_{ωβ} = {[ \frac{{\sum }_{i = 1}^{n} {ω}_{i}}{{( {\sum }_{i = 1}^{n} {ω}_{i} )}^{2} - {\sum }_{i = 1}^{n} {ω}_{i}^{2}} {\sum }_{i = 1}^{n} {( {μ}_{ωβ} - {β}_{{t}_{w}} ( i ) )}^{2} ]}^{0.5}$ |
| L2 Norm | ROC | ${τ}_{{t}_{w}} = \sqrt{{\sum }_{i = 1}^{n} {\vert {β}_{{t}_{w}} ( i ) \vert}^{2}}$ |
| Mean and Standard Deviation | Delta ROC | Tₜ_{_{w}} = μ_{Δβ} + 2 × ϑ_{Δβ} |
| Weighted Mean and Standard Deviation | Delta ROC | Tₜ_{_{w}} = μ_{ωΔβ} + ϑ_{ωΔβ} ${μ}_{ωΔβ} = \frac{{\sum }_{i = 1}^{n} {ω}_{i} {Δβ}_{{t}_{w}} ( i )}{{\sum }_{i = 1}^{n} {ω}_{i}}$ |
| | | ${ω}_{i} = 1 - \frac{\vert {μ}_{Δβ} - {Δβ}_{{t}_{w} ( i )} \vert}{max \vert {μ}_{Δβ} - {Δβ}_{{t}_{w}} \vert}$ |
| | | ${ϑ}_{ωΔβ} = {[ \frac{{\sum }_{i = 1}^{n} {ω}_{i}}{{( {\sum }_{i = 1}^{n} {ω}_{i} )}^{2} - {\sum }_{i = 1}^{n} {ω}_{i}^{2}} {\sum }_{i = 1}^{n} {( {μ}_{ωΔβ} - {β}_{{t}_{w}} ( i ) )}^{2} ]}^{0.5}$ |
| L2 Norm | Delta ROC | ${τ}_{{t}_{w}} = \sqrt{{\sum }_{i = 1}^{n} {\vert {Δβ}_{{t}_{w}} ( i ) \vert}^{2}}$ |

One approach shown in Table 2 is to ignore the prior knowledge from the standard and directly apply the estimated thresholds to detect fault. However, this may not help in achieving robustness and may still result in false alarms. Therefore, another approach includes the prior knowledge in adaptive threshold estimation. The threshold values from the standard may be added to the estimated thresholds. With prior knowledge of thresholds from the standard, the threshold may be set according to: T=τt_{ω}+τ_{standard} where τₜ_{_{ω}}={τₜ_{_{ω}}(1), τₜ_{_{ω}}(2), . . . , τₜ_{_{ω}}(n)}. τ_{standard} may represent the prior knowledge (e.g., the threshold from the standard) where n is the number of samples in a sliding time window tw that could be variable or fixed.

The statistical methods in Table 2 may be applied on ROC, β and Delta ROC, and Δβ time series whose length is determined by ta that could be fixed or variable. The delta ROC may be calculated by taking the difference between two consecutive sorted ROC either in ascending or descending order as shown in the equations: βₜ_{_{ω}}{βₜ_{_{ω}}(1), βₜ_{_{ω}}(2), . . . , βₜ_{_{ω}}(n)}, Δβₜ_{_{ω}}={Δβₜ_{_{ω}}(1), Δβₜ_{_{ω}}(2), . . . , Δₜ_{_{ω}}(n)}, where Δβₜ_{_{ω}}(i)=βₜ_{_{ω}}(i)−Δβₜ_{_{ω}}(i−1)∀i=1, 2, . . . n and where Δβₜ_{_{ω}}(1)=0, Δβₜ_{_{ω}}(2)−Δₜ_{_{ω}}(2)−Δβₜ_{_{ω}}(1), . . . , Δβₜ_{_{ω}}(n)=Δβₜ_{_{ω}}(n)−Δβₜ_{_{ω}}(n−1).

The enhanced adaptive alarms herein add a methodology for computing gas concentration gradient by selecting a time window (e.g., n=1-96 hours), computing adaptive thresholds for gas concentration gradient using one of the adaptive methods above, some of which may use prior knowledge of standards-based thresholds, and improve the fidelity of fault detection for transformers based on gas concentration and their gradients.

The above descriptions are for purposes of illustration and are not meant to be limiting. Numerous other examples, configurations, processes, etc., may exist, some of which are described in greater detail below. Example embodiments will now be described with reference to the accompanying figures.

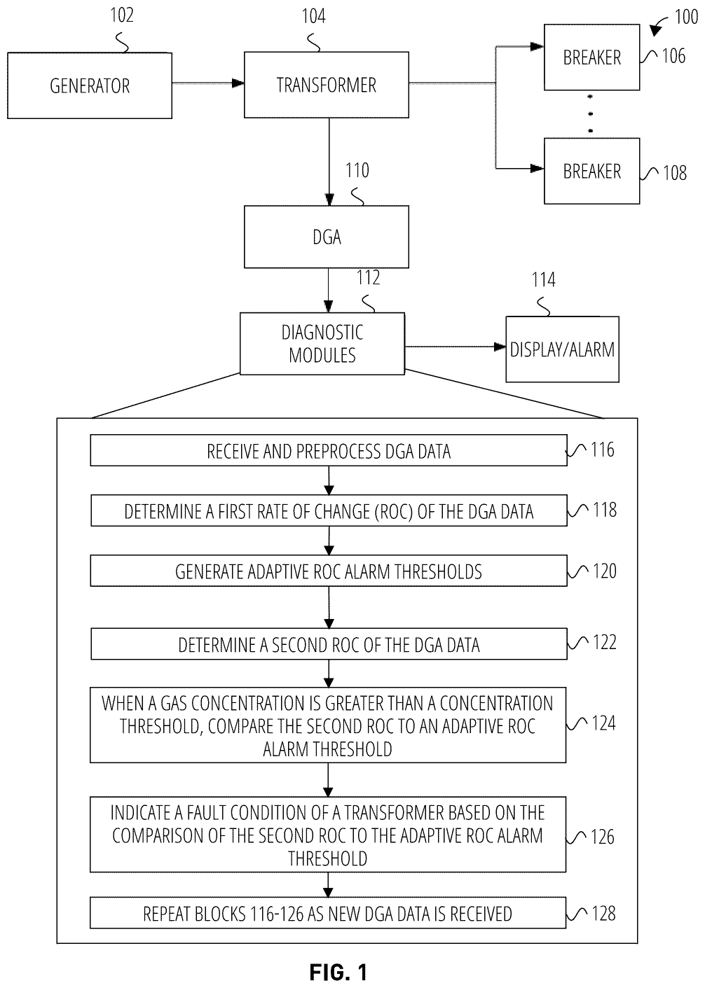

FIG. 1 illustrates an example transformer system 100 using dissolved gas analysis with adaptive alarm thresholds for rate of change in dissolved gas concentration in accordance with one embodiment of the present disclosure.

Referring to FIG. 1, the system 100 may include a generator 102 for generating and providing power to a transformer 104. The current provided by the transformer 104 may be used to operate breakers (e.g., breaker 106, breaker 108). A DGA 110 device may be coupled to the transformer 104 using an interface (e.g., one or more oil lines) to monitor and analyze any gas buildup inside of the transformer 104. The DGA 110 device may sample transformer oil periodically and execute one or more dissolved gas analysis procedures and provide DGA data to an intelligent electronic device (IED) that hosts a diagnostic modules 112. The diagnostic modules 112 may detect when DGA gas levels in the transformer 104 exceed one or more alarm thresholds, which may be fixed (e.g., static alarm thresholds from IEEE or IEC standards) or variable (e.g., adaptive threshold generated from current and historical DGA data). When the DGA gas levels exceed an alarm threshold, the diagnostic modules 112 may communicate the high DGA gas levels to a display/alarm 114 to alert a user of the gas levels, and/or may control the transformer 104 and/or breakers to prevent unsuitable operation with the unexpected gas levels in the transformer 104.

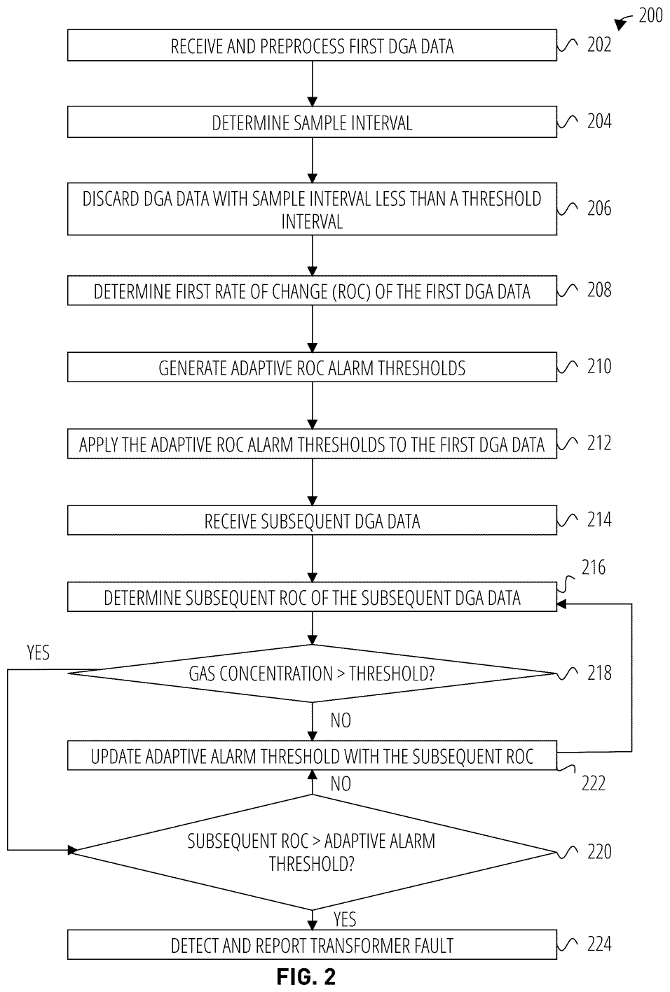

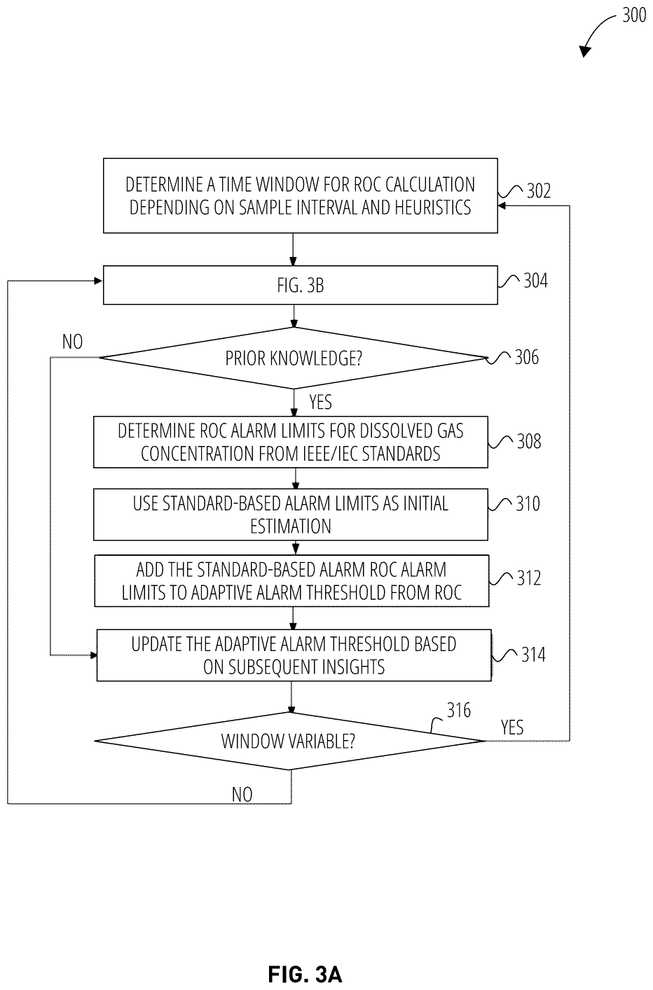

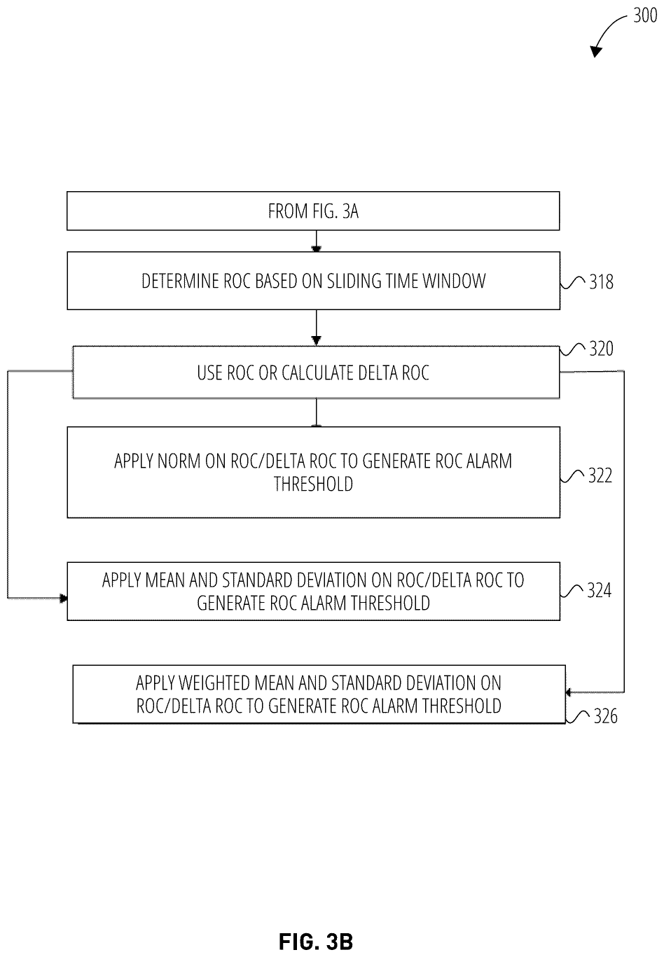

Still referring to FIG. 1, the DGA 110 device and/or the diagnostic modules 112 may generate and apply adaptive alarm thresholds on fault indicative parameters like gas concentrations, their rate of change (ROC), ratio of certain gases etc. obtained from DGA data from the transformer 104 according to a process. For example, at block 116, the diagnostic modules 112 may receive and preprocess DGA data from the DGA 110 (e.g., detecting using one or more sensors configured to detect gas levels in the transformer 104). At block 118, the diagnostic modules 112 may determine a first ROC of the DGA data over a time period. At block 120, the diagnostic modules 112 may generate one or more adaptive alarm thresholds using one of the techniques described herein (e.g., one of the techniques in Table 2). At block 124, the diagnostic modules 112 may determine a second (e.g., updated) ROC of DGA data from the transformer 104. When the gas concentration, as indicated by the updated DGA data, exceeds a concentration threshold (e.g., from the standard or learned using the adaptive techniques herein), at block 126, the diagnostic modules 112 may verify the transformer fault by comparing the second ROC to an adaptive alarm threshold for ROC. At block 127, when the second ROC exceeds the adaptive alarm threshold, the diagnostic modules 112 may detect a fault condition for the transformer 104, and may generate an alert for presentation using the display/alarm 114. At block 128, the process of block 116 through block 127 are repeated till subsequent DGA data streams to diagnostic Modules. This process is further expanded in FIGS. 2-3C.

FIG. 2 illustrates an example process 200 for using dissolved gas analysis with adaptive alarm thresholds for rate of change in dissolved gas concentration in accordance with one embodiment of the present disclosure.

Referring to FIG. 2, a device (or system, e.g., using the diagnostic modules 112 of FIG. 1) may, at block 202, receive first DGA data from a transformer (e.g., the transformer 104 of FIG. 1), and at block 204, may select a sample time. For example, transformer gases may be sampled periodically, that is at a specific time interval which could be every hour or every four hour or every day etc. At block 206, the device may sort the DGA data by sample time and discard DGA data with a sample interval less than a threshold sample interval (e.g., 50 minutes or some other time). At block 208, the device may determine a first ROC of the remaining first DGA data. The ROC may use the equation ρ=β×t+α, Where p=concentration (ppm) of target measurement, β=ROC formula, t=time (hours) from initial zero start time, α=vertical axis. Beta may be represented by

$$β = ROC = \frac{n ( {\sum }_{i} {t}_{i} {ρ}_{i} ) - ( {\sum }_{i} {t}_{i} ) ( {\sum }_{i} {ρ}_{i} )}{n ( {\sum }_{i} {t}_{i} \cdot {t}_{i} ) - {( {\sum }_{i} {t}_{i} )}^{2}}$$

and alpha may be represented by

$$α = \frac{( {\sum }_{i} {ρ}_{i} ) - β ( {\sum }_{i} {t}_{i} )}{n} ,$$

where n=number of data points, and it is determined by time window of the gas concentrations to calculate the ROC.

Still referring to FIG. 2, at block 210, the device may generate adaptive alarm thresholds for ROC, for example, using any of the techniques in Table 2 (e.g., as represented by FIGS. 3A-B3). At block 212, the device may apply the adaptive ROC alarm thresholds to the first DGA data (e.g., compare the DGA data for respective ROC in dissolved gases to respective ROC alarm thresholds) to determine whether any ROC of those gases exceeds an alarm threshold. At block 214, the device may receive subsequent DGA data for the transformer. Based on the subsequent DGA data, at block 216, the device may generate a subsequent ROC from the gas concentration in the subsequent DGA data. When a respective gas concentration is greater than a concentration threshold at block 218 (e.g., a standard-based threshold), the device may, at block 220, verify the transformer fault (e.g., as indicated by the gas concentration level exceeding the threshold) by determining whether the subsequent ROC of the gas is greater than its adaptive alarm threshold. When the respective gas concentration at block 220 is less than or equal to the standard threshold, the device may update the adaptive alarm threshold for that gas using the subsequent ROC calculated at block 216. When both subsequent gas concentration and their ROC are greater than their alarm thresholds at block 218 and 220, the device may detect a fault and report it (e.g., via the display/alarm 114 of FIG. 1).

FIG. 3A illustrates an example process 300 for using dissolved gas analysis with adaptive alarm thresholds for rate of change in dissolved gas concentration in accordance with one embodiment of the present disclosure.

At block 302, a device (or system, e.g., using the diagnostic modules 112 of FIG. 1) may determine a time window to use for ROC calculation, depending on a sample interval and heuristics. The minimum number of samples may depend on the number of samples in a window. For example, to reduce variability, the ROC time window may be set to half of the number of samples in a window. It may be desirable to set a window size with 20 or more samples.

At block 304, the process 300 may continue at FIG. 3B.

At block 306, when there is prior knowledge of the standard-based alarm thresholds, the process 300 may continue at block 308. At block 308, the device may determine ROC alarm limits for dissolved gas concentration from the IEEE/IEC standards.

At block 310, the device may apply the standard-based alarm thresholds as an initial estimation, and at block 312, the device may add the standard-based alarm thresholds to an adaptive alarm threshold from the ROC. At block 314, the device may update the adaptive alarm threshold based on subsequent insights from received DGA data. At block 316, when the window is variable, the process 300 may return to block 302. When the window is fixed, the process 300 may return to block 304.

FIG. 3B illustrates the example process 300 of FIG. 3A for using dissolved gas analysis with adaptive alarm thresholds for rate of change in dissolved gas concentration based on a sliding time window that could be fixed or variable in accordance with one embodiment of the present disclosure.

At block 318, the device may determine the gas concentration ROC based on a sliding time window. At block 320, the device may use the gas concentration ROC or may calculate a delta ROC.

At block 322, the device may apply the norm on the ROC or delta ROC to generate the ROC alarm threshold.

Alternatively, at block 324, the device may apply a mean and standard deviation on the ROC or delta ROC to generate the ROC alarm threshold.

Alternatively, at block 326, the device may apply weighted mean and standard deviation on the ROC or delta ROC to generate the ROC alarm threshold.

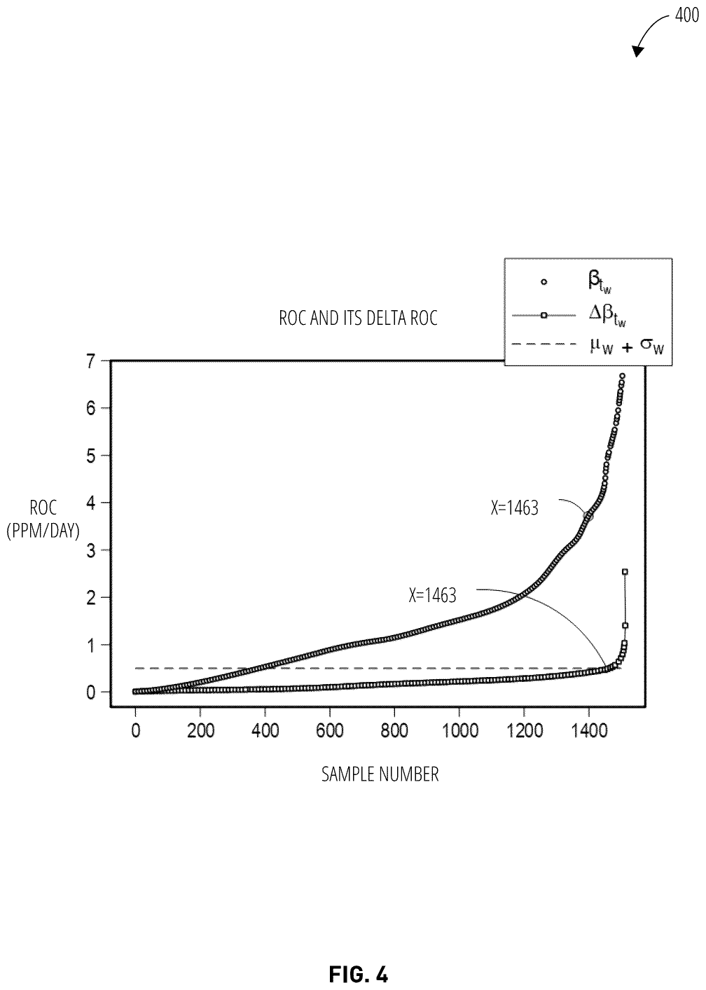

FIG. 4 illustrates an example graph 400 showing rate of change and delta rate of change of transformer gas used in dissolved gas analysis with adaptive alarm thresholds for rate of change in dissolved gas concentration in accordance with one embodiment of the present disclosure.

The graph 400 of FIG. 4 shows the methodology to calculate adaptive alarm threshold based on Δβₜ_{_{ω}} DeltaROC. The ROC represented by βₜ_{_{ω}} for the time window t_{ω}, and the delta ROC represented by Δβₜ_{_{ω}} for the time window t_{ω} per DGA sample size on the horizontal axis. The near zero slope region of DeltaROC represents the normal data, while the right-most region represents sudden change in gas concentration indicating a possible fault scenario. As shown by the graph 400, the weighted mean and standard deviation cuts the deltaROC plot at x=1463 which is the change point between near-zero and near-pi/2 slope of deltaROC. The ROC value at x=1463 represents the adaptive alarm threshold. The graph may present the change point based on the statistical methods in Table 2, here in the limit is based on the weighted mean and standard deviation of delta ROC represented by μ_{w}+θ_{w}, per DGA sample size on the horizontal axis. The ROC value, β(1463) represents the adaptive threshold and β≥β(1463) indicates the abnormal ROC in dissolved gas concentration. In this manner, the adaptive alarm threshold may be based on DeltaROC.

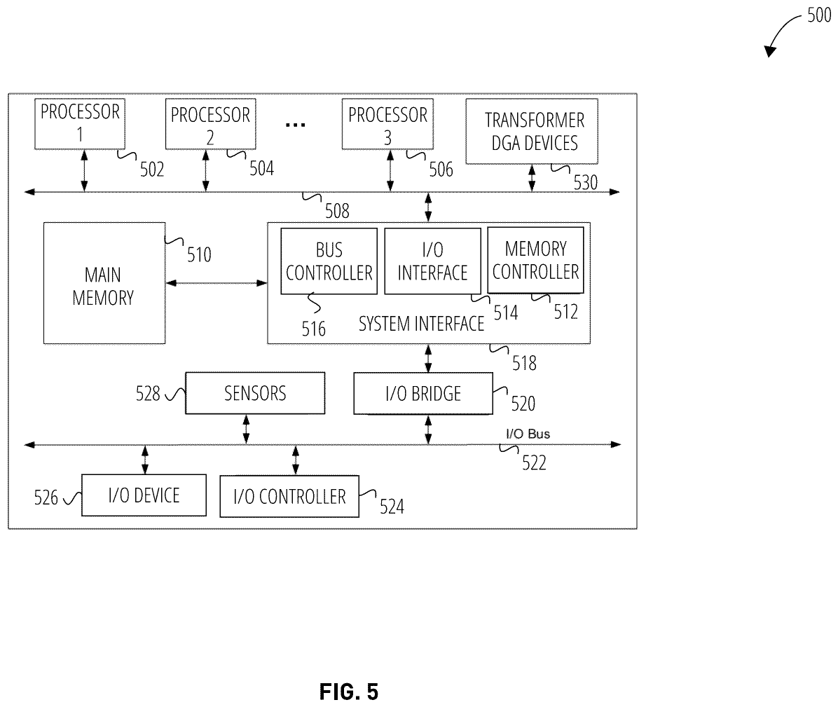

FIG. 5 is a diagram illustrating an example machine 500 that may be used in implementing embodiments of the present disclosure. For example, the machine 500 of FIG. 5 may represent at least a portion of the system 100 shown in FIG. 1, as discussed above. The machine 500 includes one or more processors 502-506. Processors 502-506 may include one or more internal levels of cache (not shown) and a bus controller 516 or bus interface unit to direct interaction with the processor bus 508. Processor bus 508, also known as the host bus or the front side bus, may be used to couple the processors 502-506 with the system interface 518. System interface 518 may be connected to the processor bus 508 to interface other components of the machine 500 with the processor bus 508. For example, system interface 518 may include a memory controller 512 for interfacing a main memory 510 with the processor bus 508. The main memory 510 typically includes one or more memory cards and a control circuit (not shown). System interface 518 may also include an input/output (I/O) I/O interface 514 to interface one or more I/O bridges I/O bridge 520 or I/O devices with the processor bus 508. One or more I/O controllers and/or I/O devices may be connected with the I/O bus 522, such as I/O controller 524 and I/O device 526, as illustrated. The machine 500 may include sensors 528, such as DGA sensors (e.g., implemented in the transformer 104 of FIG. 1) to detect gas concentrations within the transformer 104.

I/O device 526 may also include an input device (not shown), such as an alphanumeric input device, including alphanumeric and other keys for communicating information and/or command selections to the processors 502-506. Another type of user input device includes cursor control, such as a mouse, a trackball, or cursor direction keys for communicating direction information and command selections to the processors 502-506 and for controlling cursor movement on the display device.

Machine 500 may include an adaptive storage device, referred to as main memory 510, or a random access memory (RAM) or other computer-readable devices coupled to the processor bus 508 for storing information and instructions to be executed by the processors 502-506. Main memory 510 also may be used for storing temporary variables or other intermediate information during execution of instructions by the processors 502-506. Machine 500 may include a read only memory (ROM) and/or other static storage device coupled to the processor bus 508 for storing static information and instructions for the processors 502-506. The system outlined in FIG. 5 is but one possible example of a computer system that may employ or be configured in accordance with aspects of the present disclosure.

According to one embodiment, the above techniques may be performed by machine 500 in response to processor 504 executing one or more sequences of one or more instructions contained in main memory 510. These instructions may be read into main memory 510 from another machine-readable medium, such as a storage device. Execution of the sequences of instructions contained in main memory 510 may cause processors 502-506 to perform the process steps described herein. In alternative embodiments, circuitry may be used in place of or in combination with the software instructions. Thus, embodiments of the present disclosure may include both hardware and software components.

A machine readable medium includes any mechanism for storing or transmitting information in a form (e.g., software, processing application) readable by a machine (e.g., a computer). Such media may take the form of, but is not limited to, non-volatile media and volatile media and may include removable data storage media, non-removable data storage media, and/or external storage devices made available via a wired or wireless network architecture with such computer program products, including one or more database management products, web server products, application server products, and/or other additional software components. Examples of removable data storage media include Compact Disc Read-Only Memory (CD-ROM), Digital Versatile Disc Read-Only Memory (DVD-ROM), magneto-optical disks, flash drives, and the like. Examples of non-removable data storage media include internal magnetic hard disks, SSDs, and the like. The one or more memory devices may include volatile memory (e.g., adaptive random access memory (DRAM), static random access memory (SRAM), etc.) and/or non-volatile memory (e.g., read-only memory (ROM), flash memory, etc.).

Computer program products containing mechanisms to effectuate the systems and methods in accordance with the presently described technology may reside in main memory 510, which may be referred to as machine-readable media. It will be appreciated that machine-readable media may include any tangible non-transitory medium that is capable of storing or encoding instructions to perform any one or more of the operations of the present disclosure for execution by a machine or that is capable of storing or encoding data structures and/or modules utilized by or associated with such instructions. Machine-readable media may include a single medium or multiple media (e.g., a centralized or distributed database, and/or associated caches and servers) that store the one or more executable instructions or data structures.

Embodiments of the present disclosure include various steps, which are described in this specification. The steps may be performed by hardware components or may be embodied in machine-executable instructions, which may be used to cause a general-purpose or special-purpose processor programmed with the instructions to perform the steps. Alternatively, the steps may be performed by a combination of hardware, software and/or firmware.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations together with all equivalents thereof.

What is claimed is:

1. A method for using adaptive alarm thresholds for rate of change in dissolved gas concentrations for power transformer fault detection, the method comprising:
receiving, by at least one processor of a device, from at least one sensor of a power transformer, first dissolved gas data of the power transformer;
determining, by the at least one processor, a first rate of change (ROC) based on a first sliding time window of a first gas concentration of the first dissolved gas data, wherein a length of the first sliding time window is fixed or variable;
generating, by the at least one processor, based on the first ROC, a first adaptive alarm threshold for ROC with which to detect a fault in the power transformer;
receiving, by the at least one processor, from the at least one sensor, second dissolved gas data of the power transformer;
determining, by the at least one processor, a second ROC based on a second sliding time window of a second gas concentration of the second dissolved gas data, wherein a length of the second sliding time window is fixed or variable;
comparing, by the at least one processor, the second gas concentration to a static gas concentration threshold;
comparing, by the at least one processor, based on the comparison of the second gas concentration to the static gas concentration threshold, the second ROC to the first adaptive alarm threshold for ROC;
detecting, by the at least one processor, the fault based the comparison of the second gas concentration to the static gas concentration threshold and based on the comparison of the second ROC to the first adaptive alarm threshold for ROC; and
generating, by the at least one processor, an alert indicative of the fault.

2. The method of claim 1, further comprising:
receiving, from the at least one sensor, subsequent dissolved gas data of the power transformer;
determining a subsequent ROC based on a third sliding time window of a subsequent gas concentration of the subsequent dissolved gas data, wherein a length of the third sliding time window is fixed or variable;
comparing, by the at least one processor, the subsequent gas concentration to the static gas concentration threshold; and
comparing, by the at least one processor, based on the comparison of the subsequent gas concentration to the static gas concentration threshold, the subsequent ROC to the first adaptive alarm threshold for ROC,
wherein detecting the fault is further based on the comparison of the subsequent gas concentration to the static gas concentration threshold and based on the comparison of the subsequent ROC to the first adaptive alarm threshold for ROC.

3. The method of claim 1, wherein generating the first adaptive alarm threshold for ROC is further based on prior knowledge of ROC of dissolved gas concentration threshold from IEEE or IEC standards.

4. The method of claim 2, wherein generating the first adaptive alarm threshold for ROC is further based on historical patterns of the first dissolved gas data.

5. The method of claim 1, wherein generating the first adaptive alarm threshold for ROC is not based on prior knowledge of ROC of a dissolved gas concentration threshold from as IEEE or IEC standards, wherein the first sliding time window is fixed, and wherein generating first adaptive alarm threshold for ROC is further based on current and historical ROC and gas concentration from the dissolved gas data.

6. The method of claim 5, wherein the first sliding time window is variable.

7. The method of claim 6, wherein generating the first adaptive alarm threshold for ROC is further based on a norm of the first ROC.

8. The method of claim 6, wherein generating the first adaptive alarm threshold for ROC is further based on a norm of a delta of the first ROC.

9. The method of claim 6, wherein generating the first adaptive alarm threshold for ROC is further based on a mean and standard deviation of the first ROC.

10. The method of claim 6, wherein generating the first adaptive alarm threshold for ROC is further based on a mean and standard deviation of a delta of the ROC.

11. The method of claim 6, wherein generating the first adaptive alarm threshold for ROC is further based on a weighted mean and weighted standard deviation of the first ROC.

12. The method of claim 6, wherein generating the first adaptive alarm threshold for ROC is further based on a weighted mean and weighted standard deviation of a delta of the first ROC.

13. The method of claim 5, wherein the first sliding time window is fixed.

14. The method of claim 13, wherein generating the first adaptive alarm threshold for ROC is further based on a norm of the first ROC.

15. The method of claim 13, wherein generating the first adaptive alarm threshold for ROC is further based on a norm of a delta of the first ROC.

16. The method of claim 13, wherein generating the first adaptive alarm threshold for ROC is further based on a mean and standard deviation of the first ROC.

17. The method of claim 13, wherein generating the first adaptive alarm threshold for ROC is further based on a mean and standard deviation of a delta of the first ROC.

18. The method of claim 13, wherein generating the first adaptive alarm threshold for ROC is further based on a weighted mean and weighted standard deviation of the first ROC.

19. A device for using adaptive alarm thresholds for rate of change in dissolved gas concentrations for power transformer fault detection, the device comprising memory coupled to at least one processor, the at least one processor configured to:
receive, from at least one sensor of an power transformer, first dissolved gas data of the power transformer;
determine a first rate of change (ROC) based on a first sliding time window of a first gas concentration of the first dissolved gas data, wherein a length of the first sliding time window is fixed or variable;
generate, based on the first ROC, a first adaptive alarm threshold for ROC with which to detect a fault in the power transformer;
receive, from the at least one sensor, second dissolved gas data of the power transformer;
determine a second ROC based on a second sliding time window of a second gas concentration of the second dissolved gas data, wherein a length of the second sliding time window is fixed or variable;
compare the second gas concentration to a static gas concentration threshold;
compare, based on the comparison of the second gas concentration to the static gas concentration threshold, the second ROC to the first adaptive alarm threshold for ROC;
detect the fault based the comparison of the second gas concentration to the static gas concentration threshold and based on the comparison of the second ROC to the first adaptive alarm threshold for ROC; and
generate an alert indicative of the fault.

20. A system for using adaptive alarm thresholds for rate of change in dissolved gas concentrations for power transformer fault detection, the system comprising:
a dissolved gas analyzer device; and
memory coupled to at least one processor, the at least one processor configured to:
receive, from at least one sensor of an power transformer, first dissolved gas data of the power transformer;
determine a first rate of change (ROC) based on a first sliding time window of a first gas concentration of the first dissolved gas data, wherein a length of the first sliding time window is fixed or variable;
generate, based on the first ROC, a first adaptive alarm threshold for ROC with which to detect a fault in the power transformer;
receive, from the at least one sensor, second dissolved gas data of the power transformer;
determine, a second ROC based on a second sliding time window of a second gas concentration of the second dissolved gas data, wherein a length of the second sliding time window is fixed or variable;
compare the second gas concentration to a static gas concentration threshold;
compare, based on the comparison of the second gas concentration to the static gas concentration threshold, the second ROC to the first adaptive alarm threshold for ROC;
detect the fault based the comparison of the second gas concentration to the static gas concentration threshold and based on the comparison of the second ROC to the first adaptive alarm threshold for ROC; and
generate an alert indicative of the fault.
